SEQUENCE LISTING (1 sequence)

SEQ ID NO: 1 - protein, 2120 aa, synthetic construct
MPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

(19) United States
(12) Patent Application Publication
(10) Pub. No.: US 2024/0182888 A1
(43) Pub. Date: Jun. 6, 2024

(54) PE-STOP Gene Editing System and Gene Knockout Method and Application

(71) Applicant: NORTHWEST AGRICULTURAL AND FORESTRY UNIVERSITY, Xianyang (CN)

(72) Inventors: Xiaolong Wang, Xianyang (CN); Ziguo Song, Xianyang (CN); Shuhong Huang, Xianyang (CN); Bingchun Chen, Xianyang (CN); Yulin Chen, Xianyang (CN)

(73) Assignee: NORTHWEST AGRICULTURAL AND FORESTRY UNIVERSITY, Xianyang (CN)

(21) Appl. No.: 18/426,324

(22) Filed: Jan. 29, 2024

Related U.S. Application Data
(63) Continuation of application No. PCT/CN2023/082405, filed on Mar. 20, 2023.

(30) Foreign Application Priority Data
Jan. 17, 2023 (CN) CN202310060492.2

(51) Int. Cl. C12N 15/11; C12N 9/12; C12N 9/22; C12N 15/90

(52) U.S. Cl. CPC C12N 15/11; C12N 9/1276; C12N 9/22; C12N 15/907; C12Y 207/07049; C12N 2310/20

(57) ABSTRACT

The present disclosure discloses a PE-STOP gene editing system and gene knockout method and application. The gene editing system includes a prime editor protein, a pegRNA targeting a target site, and a matching nicking sgRNA for cleaving. The prime editor protein is selected from a PEmax protein, and an amino acid sequence of the PEmax is shown in SEQ ID NO. 1. The PE-STOP gene editing system can perform base replacement of a target gene sequence and introduce a termination codon in advance, thereby efficiently achieving the knockout of the target gene. The gene knockout method provided by the present disclosure has higher genotypic purity of the editing product and lower off-target activity, as well as higher genome coverage depth, therefore it has broad application prospects.

### TECHNICAL FIELD

The present disclosure relates to the field of gene editing, and in particular to a PE-STOP gene editing system, a gene knockout method and an application of gene editing system.

### BACKGROUND

Prime Editing (PE) system is a new genome editing tool based on “search and replace” developed by Anzalone et al. in 2019. This system does not require the introduction of double-strand breaks and donor DNA template in the genome of the organism being edited, to realize small segment insertion, deletion and arbitrary substitution of four bases. The earliest PE system consisted of modified guide RNA—pegRNA and prime editor protein. The pegRNA is composed of Spacer, scaffold, primer binding site (PBS) and reverse transcriptase (RT) sequence, to perform search functions, and transcribe reversely the editing information of the RT sequence to the editing site. The prime editor protein is formed by the fusion of reverse transcriptase and Cas9 nickase. This system makes up for the shortcomings of editing tools such as CRISPR/Cas9, ABE, and CBE in terms of target site limitations, frequency of indels, and number of off-target effects. It has broad application prospects in the biological field, such as studying the function of SNP through precise single base editing or studying the function of a certain gene sequences through saturation mutation. Currently, researchers have verified the effectiveness of the PE system for genome editing in plants, animal models, and human cells. Liu et al. used the PE system for the first time to establish a Hox-D13 (Hoxd13) gene editing mouse model, and Lin et al. applied this system to plant genome editing for the first time.

Introducing double-strand breaks in the target gene sequence via CRISPR-Cas9 technology can knock out the target gene through coding frame displacement. However, gene editing via double-strand breaks has been proven to be highly toxic to cells, and due to the existence of off-target activity, the risk of developing chromosome level mutations after double-strand breaks also increases significantly. The base editor can realize the targeted editing of base pair C:G to T:A or A:T to G:C in the editing window, based on the activity of APOBEC or Tad deaminase and the base mismatch repair mechanism, in cells and without double-strand breaks. Based on ABE and CBE base editors, researchers have established the i-Silence method to destroy the start codon and the iSTOP method to generate premature termination codons. Both of the above methods have been proven to be effective in efficient editing of the target site and achieving complete knockout of the target protein at the single clone level. However, the above gene knockout scheme still has certain limitations and shortcomings:

Due to the existence of the editing window, the ABE and CBE editors will edit the adjacent A or C base at the same time when editing the target site—(‘bystander editing activity’). This type of editing will cause multiple different genotypes to appear in the edited cells. During the process of disease simulation or nonsense mutation treatment, the emergence of unknown genotypes often affects the test results and treatment effects in an unpredictable way.

Due to the limitations of base mutation methods, iSTOP and i-Silence methods cannot achieve efficient coverage of the genome, and the resulting exon coverage depth limitations make it impossible for the above editing methods to perform specific translation termination for specific transcripts.

The high deamination activity of APOBEC and Tad deaminase will cause the above two methods to produce a large amount of DNA or RNA off-target editing during gene knockout, which greatly reduces the safety of their practical application.

### SUMMARY

In view of the shortcomings of existing gene knockout schemes, such as low genome coverage depth, low genotypic purity of editing products, and high off-target activity, the present disclosure provides a PE-STOP gene editing system and gene knockout methods and applications.

In a first aspect, the present disclosure provides a PE-STOP gene editing system, which includes a prime editor protein, a pegRNA targeting a target site, and a corresponding nicking sgRNA for cleaving. The prime editor protein is selected from a PEmax protein, and the PEmax has an amino acid sequence shown in SEQ ID NO.1.

In a further embodiment, the 3′ end of the pegRNA contains an anti-degradation xrRNA moiety. Compared with traditional pegRNA, anti-degradation modified xr-pegRNA can effectively resist nuclease degradation and thereby improve the editing efficiency of the PE system.

In a second aspect, the present disclosure provides an application of the gene editing system in editing genome sequences of organisms or biological cells.

In a further embodiment, the editing involves base substitution of the target gene sequence and introduction of a termination codon in advance, thereby achieving knockout of the target gene.

In a further embodiment, the number of introduced termination codons is 2-3.

In a further embodiment, the editing position of the target gene sequence comprises an NGG PAM sequence, and this editing position must be located in the first 20% of the target gene sequence.

In a further embodiment, the mutant sequence after base substitution includes TAG, TGA, and/or TAA.

In a third aspect, the present disclosure provides a method for efficiently achieving target gene knockout, which includes the following steps:
S1: constructing a plasmid containing the gene editing system according to the above embodiments based on a target gene sequence;
S2: introducing the plasmid into a biological cell, performing gene editing on the biological cell, and making a target gene undergo premature termination codon mutation.

Compared with the prior art, the present disclosure has the following beneficial effects:

The present disclosure achieves efficient introduction of target site termination codons by combining codon-optimized PEmax and anti-degradation modified xr-pegRNA. This gene knockout method has higher genotypic purity of the editing product and lower off-target activity, as well as higher genome coverage/coverage depth, therefore this method has broad application prospects.

Compared with the existing iSTOP and i-Silence systems, the gene knockout method of the present disclosure gets removes editing window and editing method limitations, can significantly improve the coverage of the gene knockout scheme in the genome, and it is beneficial to the application of subsequent high throughput screening tests. This method is based on the biological process of reverse transcription to introduce premature termination codons into the genome sequence, it will not cause bystander editing at the editing site. Therefore, compared with currently commonly used gene knockout methods, this method has better genotype purity, and it is beneficial to the development of disease models and the therapeutic application of nonsense mutations. The PE-STOP method relies on PE technology and almost no off-target activity is detected at the DNA level and RNA level, proving that this method is safer than previous gene knockout schemes. The proposal of the present disclosure can further promote the application of gene knockout methods and has broad application prospects.

### BRIEF DESCRIPTION OF THE DRAWINGS

FIG. 1 is a schematic diagram of introduction of a termination codon via PE-STOP editing.

FIG. 2 shows that PE-STOP can efficiently convert different amino acids into termination codons.

FIG. 3 shows that PE-STOP has higher ORF and exon coverages in the human genome. A: PE-STOP has higher ORF coverage than iSTOP and i-Silence; B: PE-STOP has higher exon coverage than iSTOP.

FIG. 4 shows construction of a monoclonal PD1 knockout N2a cell line via PE-STOP editing. A: Deep sequencing identifies the editing efficiency of PE-STOP; B: Sanger sequencing identifies PD1 homozygous editing monoclonal cells; C: Western blot verifies complete knockout of PD1 protein.

FIG. 5 shows a genotypic purity comparison at five editing sites in HEK293T cells between PE-STOP and iSTOP and i-Silence methods.

FIG. 6 shows that PE-STOP has lower off-target activity. A: PE-STOP off-target editing is not detected at predicted off-target DNA sites; B: Based on transcriptome sequencing, PE-STOP off-target editing is not detected at the RNA level.

### DETAILED DESCRIPTION OF EMBODIMENTS

The present disclosure is set forth in detail below with reference to the accompanying drawings and specific embodiments, but the disclosed embodiments should not be understood as limitations of the present disclosure. Unless otherwise specified, the technical means used in the following embodiments are conventional means well known to those skilled in the art. The materials, reagents, etc. used in the following embodiments can all be obtained from commercial sources, unless otherwise specified.

### Vector (Carrier) Information

pCMV-PEmax vector: Addgene, Cat. No.: 174820;
pCMV-AncBE4max vector: Addgene, Cat. No.: 112094;
pCMV-ABE8e vector: Addgene, Cat. No.: 138489;
pGL3-U6-sgRNA-EGFP vector: Addgene, Cat. No.: 107721;
pGL3-U6-sgRNA-mCherry vector: prepared in the laboratory, published in “Efficient generation of mouse models with the prime editing system”.

### Embodiment 1. PE-STOP Mutates Different Types of Amino Acids Into Termination Codons in HEK 293T Cells

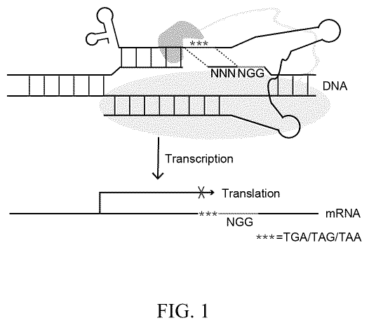

A total of 15 sites in four genes, namely PRNP, RNF2, RIT1, and ALDOB, are selected as target editing sites in the HEK 293T cell genome, and PE-STOP is used to carry out premature termination codon mutation (the process is shown in FIG. 1).

### I. Preparation of Components of PE-STOP Targeting Target Sites

1. Construct Nicking sgRNA Expression Vector Targeting the Target Sites

TABLE 1
Preparation of sgRNA oligonucleotide
sequences targeting target sites

| | Oligo- | |
|---|---|---|
| | nucleotide | |
| Target | chain | Oligonucleotide |
| gene | name | sequence |
| ALDOB-T | Forward1 | accgCAAAGGACAGTATGTTCACA |
| | Reverse1 | aaacTGTGAACATACTGTCCTTTG |
| ALDOB-Q | Forward2 | accgAGGCAGACAGGGTCAAGGTG |
| | Reverse2 | aaacCACCTTGACCCTGTCTGCCT |
| ALDOB-L | Forward3 | accgGTCTGGTGGCATGAGTGAAG |
| | Reverse3 | aaacCTTCACTCATGCCACCAGAC |
| ALDOB-E | Forward4 | accgTCCTGCAGCTGTTCCTGGTA |
| | Reverse4 | aaacTACCAGGAACAGCTGCAGGA |
| ALDOB-N | Forward5 | accgGGTCCTGGCTGCTGTCTACA |
| | Reverse5 | aaacTGTAGACAGCAGCCAGGACC |
| ALDOB-D | Forward6 | accgCTGCCAGTATGTTACTGAGA |
| | Reverse6 | aaacTCTCAGTAACATACTGGCAG |
| ALDOB-G | Forward7 | accgAATATCCTTACCTTGAATGG |
| | Reverse7 | aaacCCATTCAAGGTAAGGATATT |
| ALDOB-I | Forward8 | accgAAAACACTGAAGAGAACCGC |
| | Reverse8 | aaacGCGGTTCTCTTCAGTGTTTT |
| PRNP-C | Forward9 | accgGAGGCCCAGGTCACTCCATG |
| | Reverse9 | aaacCATGGAGTGACCTGGGCCTC |
| PRNP-A | Forward10 | accgCGGCTTGTTCCACTGACTGT |
| | Reverse10 | aaacACAGTCAGTGGAACAAGCCG |
| PRNP-Y | Forward11 | accgAGTACACTTGGTTGGGGTAA |
| | Reverse11 | aaacTTACCCCAACCAAGTGTACT |
| PRNP-F | Forward12 | accgTTACCAGAGAGGATCGAGCA |
| | Reverse12 | aaacTGCTCGATCCTCTCTGGTAA |
| RIT1-W | Forward13 | accgTTATAAGCATCTTCTACAGG |
| | Reverse13 | aaacCCTGTAGAAGATGCTTATAA |
| RNF2-K | Forward14 | accgTCTAGATACATAAAGACTTC |
| | Reverse14 | aaacGAAGTCTTTATGTATCTAGA |
| RNF2-P | Forward15 | accgATCAAGAGAGAGTATTAGCC |
| | Reverse15 | aaacGGCTAATACTCTCTCTTGAT |

(2) Anneal nicking sgRNA oligonucleotide chain to obtain nicking sgRNA annealing product. The annealing system and procedure are as follows:

### Annealing System:

| | |
|---|---|
| Forward (100 μM) | 5 μL |
| Reverse (100 μM) | 5 μL |
| Total | 10 μL |

### Annealing Procedure:

| 95° C. | 5 min | |
|---|---|---|
| 98° C. to 85° C. | −2° | C./cycle |
| 85° C. to 25° C. | −0.1° | C./cycle |

(3) Digest pGL3-U6-sgRNA-mCherry using BsaI restriction endonuclease at 37° C. for 8 hours. The digestion system is as follows:

| | | |
|---|---|---|
| pGL3-U6-sgRNA-mCherry | 2000 | ng |
| 10 × CutSmart Buffer | 5 | μL |
| Bsal-HFV2 | 1 | μL |

| | |
|---|---|
| ddH₂O | Add to 50 μL |

(4) Purify and recover the linearized pGL3-U6-sgRNA-mCherry, and ligate it with the sgRNA annealing product at 16° C. overnight. The ligation system is as follows:

| | | |
|---|---|---|
| 5 × T4 DNA Ligase Buffer | 2 | μL |
| T4 DNA Ligase | 1 | μL |
| sgRNA annealing product | 5 | μL |
| Digested pGL3-U6-sgRNA-mCherry | 50 | ng |

| | |
|---|---|
| ddH₂O | Add to 10 μL |

(5) Transform the ligation product into DH5α competent cells, and pick single clones for sequencing the next day. Expand and culture the bacteria solution with correct sequencing results and extract the plasmid to obtain the nicking sgRNA expression plasmid targeting the target sites.

2. Construct an xr-pegRNA Expression Vector Targeting the Target Sites

(1) Design and synthesize spacer, RT-PBS and scaffold oligonucleotides in xr-pegRNA targeting 15 target sites (Table 2 and Table 3). The lowercase letters in the sequences are adapter (linker) parts.

TABLE 2
Oligonucleotide sequences of spacer and RT-PBS at different sites

| Target | pegRNA | oligonucleotide | |
|---|---|---|---|
| gene | component | chain name | oligonucleotide chain sequence |
| ALDOB-T | Spacer | Forward16 | accgGGCTGTGAAGAGCGACTGGGgtttc |
| | | Reverse16 | ctctgaaacCCCAGTCGCTCTTCACAGCC |
| | RT-PBS | Forward17 | gtgcAGTCGCTCTTCACTGGGGCTGCTTCCTCAC |
| | | Reverse17 | gacaGTGAGGAAGCAGCCCCAGTGAAGAGCGACT |
| ALDOB-Q | Spacer | Forward18 | accgCATACTGTCCTTTGGCCGCCgtttc |
| | | Reverse18 | ctctgaaacGGCGGCCAAAGGACAGTATG |
| | RT-PBS | Forward18 | gtgcGGCCAAAGGACAGCTAGGCTAACTGCTCAGC |
| | | Reverse18 | gacaGCTGAGCAGTTAGCCTAGCTGTCCTTTGGCC |
| ALDOB-L | Spacer | Forward19 | accgGGCAAAGGTTGATAGCATTGgtttc |
| | | Reverse19 | ctctgaaacCAATGCTATCAACCTTTGCC |
| | RT-PBS | Forward20 | gtgcTGCTATCAACCTTTGCCACTCTCAACTTAAA |
| | | Reverse20 | gacaTTTAAGTTGAGAGTGGCAAAGGTTGATAGCA |
| ALDOB-E | Spacer | Forward21 | accgGTGGCCATAGCTACTTGTTCgtttc |
| | | Reverse21 | ctctgaaacGAACAAGTAGCTATGGCCAC |
| | RT-PBS | Forward22 | gtgcCAAGTAGCTATGGAGTATACTCCATCA |
| | | Reverse22 | gacaTGATGGAGTATACTCCATAGCTACTTG |
| ALDOB-N | Spacer | Forward23 | accgATGTCCAGCAGTCACCATGTgtttc |
| | | Reverse23 | ctctgaaacACATGGTGACTGCTGGACAT |
| | RT-PBS | Forward24 | gtgcTGGTGACTGCTGGCCTGCTAAAGCCCTCAA |
| | | Reverse24 | gacaTTGAGGGCTTTAGCAGGCCAGCAGTCACCA |
| ALDOB-D | Spacer | Forward25 | accgTCCAGGTCATGGTCTCCATCgtttc |
| | | Reverse25 | ctctgaaacGATGGAGACCATGACCTGGA |
| | RT-PBS | Forward26 | gtgcGGAGACCATGACCAGGTAATTCCTTCA |
| | | Reverse26 | gacaTGAAGGAATTACCTGGTCATGGTCTCC |
| ALDOB-G | Spacer | Forward27 | accgTATCCACAGTTAGACCAAGGgtttc |
| | | Reverse27 | ctctgaaacCCTTGGTCTAACTGTGGATA |
| | RT-PBS | Forward28 | gtgcTGGTCTAACTGTGAGAGGAGCACCTGAT |
| | | Reverse28 | gacaATCAGGTGCTCCTCTCACAGTTAGACCA |
| ALDOB-I | Spacer | Forward29 | accgACCCCCGATGCTCTGGTTGAgtttc |
| | | Reverse29 | ctctgaaacTCAACCAGAGCATCGGGGGT |
| | RT-PBS | Forward30 | gtgcACCAGAGCATCGGTGTGGACAGTTCCTCAA |
| | | Reverse30 | gacaTTGAGGAACTGTCCACACCGATGCTCTGGT |
| PRNP-C | Spacer | Forward31 | accgTTATGGCGAACCTTGGCTGCgtttc |
| | | Reverse31 | ctctgaaacGCAGCCAAGGTTCGCCATAA |
| | RT-PBS | Forward32 | gtgcGCCAAGGTTCGCCACCAGCATCCATCA |
| | | Reverse32 | gacaTGATGGATGCTGGTGGCGAACCTTGGC |
| PRNP-A | Spacer | Forward33 | accgACCAACATGAAGCACATGGCgtttc |
| | | Reverse33 | ctctgaaacGCCATGTGCTTCATGTTGGT |
| | RT-PBS | Forward34 | gtgcATGTGCTTCATGTCTGCTGCAGCACCTCAC |
| | | Reverse34 | gacaGTGAGGTGCTGCAGCAGACATGAAGCACAT |
| PRNP-Y | Spacer | Forward35 | accgACATTTCGGCAGTGACTATGgtttc |
| | | Reverse35 | ctctgaaacCATAGTCACTGCCGAAATGT |
| | RT-PBS | Forward36 | gtgcAGTCACTGCCGAAAGTAACGGTCCTCCT |
| | | Reverse36 | gacaAGGAGGACCGTTACTTTCGGCAGTGACT |
| PRNP-F | Spacer | Forward37 | accgACCTTCCTCATCCCACTATCgtttc |
| | | Reverse37 | ctctgaaacGATAGTGGGATGAGGAAGGT |
| | RT-PBS | Forward38 | gtgcAGTGGGATGAGGACTTTCCTCATCTAACTGAT |
| | | Reverse38 | gacaATCAGTTAGATGAGGAAAGTCCTCATCCCACT |
| RIT1-W | Spacer | Forward39 | accgTGATGATGAGCCTGCCAATCgtttc |
| | | Reverse39 | ctctgaaacGATTGGCAGGCTCATCATCA |
| | RT-PBS | Forward40 | gtgcTGGCAGGCTCATCATCCAAAATGTCTAGAT |
| | | Reverse40 | gacaATCTAGACATTTTGGATGATGAGCCTGCCA |
| RNF2-K | Spacer | Forward41 | accgTAACCTCACAGCCAGATACTgtttc |
| | | Reverse41 | ctctgaaacAGTATCTGGCTGTGAGGTTA |
| | RT-PBS | Forward42 | gtgcATCTGGCTGTGAGTGATCACTTATCCTCAT |
| | | Reverse42 | gacaATGAGGATAAGTGATCACTCACAGCCAGAT |
| RNF2-P | Spacer | Forward43 | accgGCTTCATACTCATCACGACTgtttc |
| | | Reverse43 | ctctgaaacAGTCGTGATGAGTATGAAGC |
| | RT-PBS | Forward44 | gtgcCGTGATGAGTATGATCAGCAAAATTTATTCAAGT |
| | | Reverse44 | gacaACTTGAATAAATTTTGCTGATCATACTCATCACG |

TABLE 3
Scaffold oligonucleotide sequences

| | oligo- | |
|---|---|---|
| | nucleotide | |
| pegRNA | chain | oligonucleotide |
| component | name | chain sequence |
| scaffold | Forward45 | AGAGCTAGAAATAGCAAGT |
| | | TGAAATAAGGCTAGTCCGT |
| | | TATCAACTTGAAAAAGTGG |
| | | CACCGAGTCG |
| | Reverse46 | GCACCGACTCGGTGCCACT |
| | | TTTTCAAGTTGATAACGGA |
| | | CTAGCCTTATT |
| | | TCAACTTGCTATTTCTAG |

(2) Prepare Buffer used for annealing. The formula is as follows:

| | | |
|---|---|---|
| NaCl | 0.08766 | g |
| 10 mM Tris-HCl Buffer (pH = 8.5) | 0.2 | mL |
| ddH₂O | 30 | mL |

(3) Anneal the forward and reverse chains of the scaffold oligonucleotide chain to obtain the scaffold annealing product. The annealing procedure is the same as the sgRNA annealing procedure. The annealing system is as follows:

| | |
|---|---|
| Forward (100 μM) | 1 μL |
| Reverse (100 μM) | 1 μL |
| annealing Buffer | 23 μL |
| Total | 25 μL |

(4) Anneal the spacer and RT-PBS oligonucleotide chains respectively to obtain spacer annealing product and RT-PBS annealing product respectively. The annealing system and procedure are as follows:

### Annealing System:

| | |
|---|---|
| Oligonucleotide sequence forward (10 μM) | 1 μL |
| Oligonucleotide sequence reverse (10 μM) | 1 μL |
| Annealing Buffer | 2 μL |
| ddH₂O | 6 μL |
| Total | 10 μL |

### Annealing Procedure:

| | | |
|---|---|---|
| 95° C. | 5 | min |
| 95° C. | 30 | s |

| | | |
|---|---|---|
| 85° C. to 25° C. | −1° C./cycle | 60 cycles |
| 25° C. to 5° C. | −2° C./cycle | 10 cycles |
| 4° C. | Forever | |

(5) Using pGL3-U6-sgRNA-EGFP plasmid as a template, use a PCR method to obtain pGL3-U6-xr-pegRNA-EGFP (xr-pegRNA) expression plasmid. The specific operations are as follows:

a. Use F primer: 5′-agctaggtctcctgtcaggcctgctagtcagccacagtttgg-3′, R primer: 5′-tctctcggtctcacggtgtttcgtcctttccac-3′ to amplify pGL3-U6-sgRNA-EGFP, linearize it and use Bsa I enzyme to perform a single enzyme digestion reaction to create sticky end.

### PCR Amplification System:

| | | |
|---|---|---|
| 2 × Phanta Flash Master Mix (Dye Plus) | 12.5 | μL |
| Template plasmid | 1 | μL |
| F (10 μM) | 0.5 | μL |
| R (10 μM) | 0.5 | μL |
| ddH₂O | 10.5 | μL |
| Total | 25 | μL |

### PCR Amplification Procedure:

| 98° C. | 3 | min | | |
|---|---|---|---|---|
| 98° C. | 10 | s | | |
| 58° C. | 5 | s | | 25 cycles |
| 72° C. | 45 | s | {close oversize brace} | |
| 72° C. | 5 | min | | |

| | |
|---|---|
| 4° C. | Forever |

b. Purify and recover backbone of xr-pegRNA expression plasmid after enzyme digestion, and use T4 DNA Ligase to respectively ligate spacer annealing product, RT-PBS annealing product and scaffold annealing product targeting the same target site with backbone of xr-pegRNA expression plasmid overnight at 16° C. The ligation system is as follows:

| | | |
|---|---|---|
| 5 × T4 DNA Ligase Buffer | 2 | μL |
| T4 DNA Ligase | 0.5 | μL |
| PBS-RT annealing product | 2 | μL |
| Spacer annealing product | 2 | μL |
| Scaffold annealing product | 2 | μL |
| Plasmid template backbone after enzyme | 30 | ng |
| digestion and recovery | | |
| ddH₂O | Add to 10 | μL |

c. Transform the ligation product into DH5α competent cells, and pick single clones for Sanger sequencing the next day. Expand and culture the bacteria solution with correct sequencing results and extract the plasmid. The obtained plasmid is xr-pegRNA expression plasmid targeting the target sites.

### 3. Prepare the PE Protein Expression Plasmid

Transform the pCMV-PEmax vector into DH5α competent cells, and pick single clones for Sanger sequencing the next day. Expand and culture a bacterial clone with correct sequencing results and extract the plasmid. The obtained plasmid is the PEmax protein (the amino acid sequence is shown in SEQ ID NO. 1) expression plasmid.

### II. PE-STOP Mutates Multiple Different Types of Amino Acids in HEK 293T Cells.

The PE-STOP components targeting the same site are transiently transfected and the editing efficiency is measured.

### 1. Seeding Cells

Resuscitate and culture the frozen HEK 293T cells in a 10 cm culture dish, add 12 mL of complete culture medium (90% high glucose DMEM+10% fetal calf serum+working concentration of penicillin-streptomycin), and incubate at 37° C. and under CO₂ 5% condition. When the cell density reaches 90%, the cells are seeded into a 24-well plate and cultured continuously.

### 2. Cell Transfection and Sorting

(1) When the cell density in the 24-well plate reaches 75%˜85%, use EZ trans transfection reagent to co-transfect the expression plasmids of each component of the PE system into the cells according to the instruction. The transfection mixture system is as follows:

| | |
|---|---|
| pCMV-PEmax | 900 ng |
| pegRNA expression plasmid | 300 ng |
| sgRNA expression plasmid | 100 ng |
| EZ trans transfection reagent | 2.5 μL |
| DMEM culture solution | 100 μL |

(2) Let the mixed system stand at room temperature for 10 minutes;

(3) Add the above mixed transfection solution to the cells in each well;

(4) After 8 hours of transfection, remove the culture solution containing the transfection reagent and add 700 μL of complete culture medium;

(5) After 72 hours of transfection, remove the culture solution, wash the cells in each well with 200 μL PBS solution, then digest and collect the cells into a 1.5 mL centrifuge tube, and resuspend the cell pellet in 260 μL PBS solution;

(6) Filter the resuspended cells to form a single cell suspension and add it into a flow tube, use a flow cytometer to perform FACS sorting, and collect 10,000˜20,000 cells with the top 20% of GFP fluorescence intensity.

### 3. Detection of PE-STOP Editing Efficiency

Centrifuge the above collected cells and add cell lysis solution for lysis, use Phanta Super-Fidelity DNA Polymerase to amplify the DNA sequence containing the target sites. The PCR reaction system and condition are as follows:

| | | |
|---|---|---|
| Buffer | 12.5 | μL |
| dNTP Mix | 0.5 | μL |
| Primer F | 0.5 | μL |
| Primer R | 0.5 | μL |
| Phanta Super-Fidelity DNA Polymerase | 0.5 | μL |
| Cell lysis solution | 1-2 | μL |
| ddH₂O | Add to 25 | μL |

### PCR Procedure:

| | | | | |
|---|---|---|---|---|
| 95° C. | 5 | min | | |
| 95° C. | 15 | s | | |
| 68° C. | −0.2° | C./cycle | | |
| 72° C. | 10 | s | {close oversize brace} | 25 cycles |
| 72° C. | 5 | min | | |

| | |
|---|---|
| 4° C. | Forever |

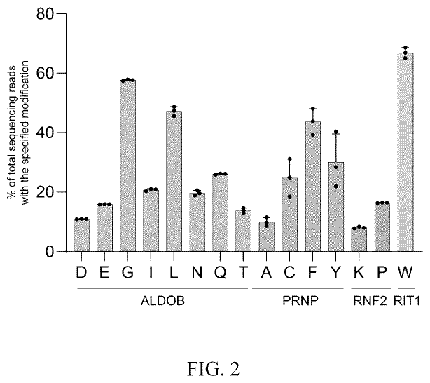

The PCR products are purified and recovered after the band specificity is detected by agarose gel electrophoresis, and then Sanger sequencing and targeted deep sequencing are performed respectively, and the editing efficiency is analyzed. The results are shown in FIG. 2, indicating that the PE-STOP system can effectively mutate different types of amino acids to termination codons in cell lines, highlighting the flexibility of this gene knockout method.

### Embodiment 2. Establishment of PD1 Knockout N2a Cell Line by PE-STOP

Taking the PD1 gene in the N2a cell genome as the target site, PE-STOP is used to design different pegRNA to introduce termination codons and single clone cells are screened to establish a PD1 knockout N2a cell line, proving the feasibility of PE-STOP in establishing a knockout cell line.

### I. Preparation of Various Components of PE-STOP Targeting N2a Cell Target Site and Detection of Deep-Seq Efficiency

1. Prepare Nicking sgRNA Vectors Targeting Different Sites

Design and synthesize oligonucleotide sequences of nicking sgRNA targeting two different positions in the PD1 CDS region (Table 5), where the lowercase letters represent the adapter (linker) part.

TABLE 5
Preparation of sgRNA oligonucleotide
sequences targeting target sites

| | Oligo- | |
|---|---|---|
| Target | nucleotide | |
| gene | chain name | Sequence |
| PD1-site1 | Forward47 | accgCCAATTGATCCCACATCCCT |
| | Reverse48 | aaacAGGGATGTGGGATCAATTGG |
| PD1-site2 | Forward49 | accgTGTCATTGCGCCGTGTGTCA |
| | Reverse50 | aaacTGACACACGGCGCAATGACA |

The experimental procedures related to the annealing and ligation of the Nicking sgRNA vector and the subsequent extraction of plasmids are consistent with Embodiment 1.

2. Prepare xr-pegRNA Vectors Targeting Different Sites and Measure Editing Efficiency

The oligonucleotide sequences of spacer and RT-PBS targeting 2 sites are designed and synthesized (Table 6), where the lowercase letters in the sequence are the adapter (linker) parts.

TABLE 6
Preparation of oligonucleotide sequences
of spacer and RT-PBS targeting
target sites

| | | Oligo- | |
|---|---|---|---|
| Target | pegRNA | nucleotide | oligonucleotide |
| gene | component | chain name | chain sequence |
| PD1-site1 | Spacer | Forward51 | accgAGGTACCCTG |
| | | | GTCATTCACTgttt |
| | | | c |
| | | Reverse52 | ctctgaaacAGTGA |
| | | | ATGACCAGGGTACC |
| | | | T |
| | RT-PBS | Forward53 | gtgcGAATGACCAG |
| | | | GGTACTGCAGCACA |
| | | | GCTCAAGT |
| | | Reverse54 | gacaACTTGAGCTG |
| | | | TGCTGCAGTACCCT |
| | | | GGTCATTC |
| PD1-site2 | Spacer | Forward55 | accgAGATCATACA |
| | | | GCTGCCCAACgttt |
| | | | c |
| | | Reverse56 | ctctgaaacGTTGG |
| | | | GCAGCTGTATGATC |
| | | | T |
| | RT-PBS | Forward57 | gtgcGGGCAGCTGT |
| | | | ATGATGTGGAAGTC |
| | | | ATGTCAGTT |
| | | Reverse58 | gacaAACTGACATG |
| | | | ACTTCCACATCATA |
| | | | CAGCTGCCC |

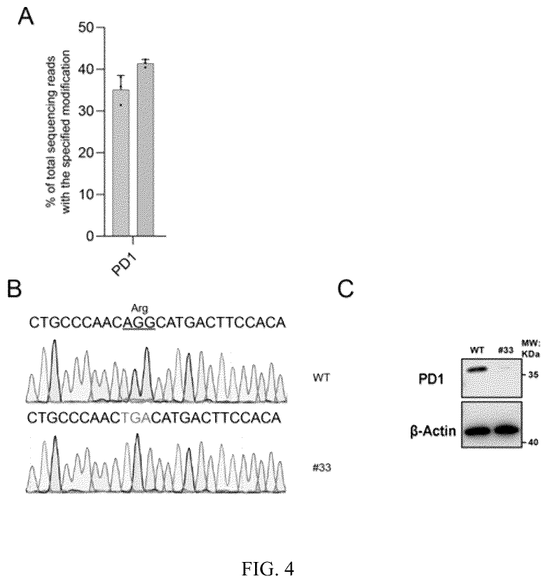

The construction method of the xr-pegRNA expression plasmid targeting the two sites and the subsequent site-directed mutation efficiency detection method in N2a cells are consistent with Embodiment 1. The results are shown in FIG. 4A, indicating that PE-STOP can efficiently generate premature termination codons in the N2a cell line.

### II. Establishment of PD1 Knockout Cell Line

### Sorting and Culturing of Monoclonal Cells

After transient transfection of N2a cells, monoclonal cells are obtained based on flow sorting technology and the cells are expanded and cultured for later identification of gene editing efficiency and establishment of knockout cell lines.

Preparation of N2a cells: resuscitate and culture the frozen N2a cells in a 10 cm culture dish, add 12 mL of complete culture medium (90% high glucose DMEM+10% fetal bovine serum+working concentration of penicillin-streptomyces), culture them at 37° C., CO₂ 5%. When the cell density reaches 90%, seed the cells into a 6-well plate and continue culturing.

When the cell density in the 6-well plate reaches 50%˜60%, use EZ trans transfection reagent to co-transfect the expression plasmids of each component of the PE system into the cells according to the instruction. The transfection mixture system is as follows:

| | | |
|---|---|---|
| pCMV-PEmax | 2700 | ng |
| pegRNA expression plasmid | 900 | ng |
| sgRNA expression plasmid | 300 | ng |
| EZ trans transfection reagent | 10 | μL |
| DMEM culture solution | 700 | μL |

(3) Let the mixed system stand at room temperature for 10 minutes;

(4) Add the above mixed transfection solution to the cells in each well;

(5) After 8 hours of transfection, remove the culture solution containing the transfection reagent and add 3 mL of complete culture medium;

(6) After 48 hours of transfection, remove the culture solution, wash the cells in each well with 500 μL PBS solution, then digest and collect the cells into a 1.5 mL centrifuge tube, and resuspend the cell pellet in 600 μL PBS solution;

(7) Filter the resuspended cells through a cell sieve (4.5 μM) to form a single cell suspension and add it to a flow tube, use a flow cytometer to perform 96-well plate FACS monoclonal sorting and classify and select those with the top 50% of GFP fluorescence intensity as the cell population, add 200 μL DMEM culture solution (15% fetal calf serum+working concentration of penicillin-streptomycin) to the 96-well plate that receives the monoclonal cells in advance, and then place it in the incubator to continue culturing for 10-14 days after sorting.

### 2. Identification of PD1 Knockout Monoclonal Cells

(1) Identify homozygous editing cells, observe each well under a microscope after culturing the cells in the 96-well plate for 10-14 days, and mark the wells with cell clones with a marker for subsequent identification of editing efficiency.

(2) Discard the culture solution in the marked wells, add 20 μL trypsin for digestion, digest for 30 s, add 200 μL culture solution to terminate digestion, use a pipette to blow and beat the cells in each well, and draw 50 μL liquid to the PCR tube, mark clone numbers sequentially for genome extraction and identification, transfer the remaining liquid to a 48-well culture plate, and add 400 μL of culture solution to continue culturing.

(3) Centrifuge the liquid in the PCR tube, discard most of the culture solution, add 40 μL of cell lysis buffer to extract the genome, and draw 3 μL of the genome stock solution for PCR amplification to identify homozygous editing.

### PCR Reaction System:

| | | |
|---|---|---|
| Buffer | 12.5 | μL |
| dNTP Mix | 0.5 | μL |
| Primer F | 0.5 | μL |
| Primer R | 0.5 | μL |
| Phanta Super-Fidelity DNA Polymerase | 0.5 | μL |
| cell lysis buffer | 3 | μL |
| ddH₂O | Add to 25 | μL |

### PCR Procedure:

| | | | | |
|---|---|---|---|---|
| 95° C. | 5 | min | | |
| 95° C. | 15 | s | | |
| 68° C. | −0.2° | C./cycle | | |
| 72° C. | 10 | s | {close oversize brace} | 35 cycles |
| 72° C. | 5 | min | | |

| | |
|---|---|
| 4° C. | Forever |

(4) The PCR product is purified and recovered after the band specificity is detected by agarose gel electrophoresis, and then identified by Sanger sequencing.

(5) For the identified homozygous cells, label them in a 48-well plate, replace with 500 μL of fresh culture solution and continue to culture for 5-7 days. The remaining 50% edited cells and WT cells are discarded.

(6) Cell passage can be carried out after the confluence of the homozygous editing cells reaches more than 80%. Add 40 μL trypsin to the cells in each well for digestion, digest for 30 s-1 min, add 400 μL culture solution to terminate the digestion, transfer to a 12-well plate after gently blowing and beating, add 1 mL of culture solution and place them in a cell incubator to continue culturing for 3-6 days.

(7) When the cell confluence reaches more than 80%, freeze the cell line and extract the protein, add 100 μL trypsin to the cells in each well for digestion, digest for 30 s-1 min; add 1 mL culture medium to terminate the digestion, gently pipet (blow and beat) and draw 500 μL respectively and transfer into two 1.5 mL centrifuge tubes for centrifugation (800 g, 4 min), discard the supernatant in one tube and add 1 mL of cell cryopreservation solution prepared in advance to freeze the cell line, use another tube for protein extraction to perform follow-up WB test.

(8) When extracting proteins, add protease inhibitors to the RIPA lysis buffer at a ratio of 1:100 in advance, absorb 100 μL of the prepared RIPA lysis buffer, resuspend the centrifuged cells, and lyse them on ice for 30 minutes; after lysis is complete, put it into a 4° C. centrifuge for centrifugation (12000 rpm, 30 min); after the centrifugation is completed, absorb the supernatant and transfer it to a new 1.5 mL centrifuge tube to obtain the total cell protein.

(9) BCA protein quantification: refer to the instruction of the Thermo BCA quantification kit to quantify the total protein extracted from KO cells and WT cells. The specific method is as follows: Mix A solution and B solution in advance according to the ratio of 1:50, and add 200 μL of mixed solution to each well of a 96-well plate, and perform gradient dilution of the standard solution to obtain standard samples of 2 mg/mL, 1.5 mg/mL, 1 mg/mL, 0.5 mg/mL, 0.25 mg/mL, and 0 mg/mL. Then add 4 μL of standard solution and sample (2 replicates) to the mixed solution in each well, and place the 96-well plate in a 37° C. incubator and incubate for 25 min. After the incubation is completed, use a microplate reader to measure the absorbance of each well at 562 nm. Fit and linearly predict the data of different concentration gradient of standard solutions to calculate the total protein amount of the sample, aspirate the sample according to 12 μg of the protein loading amount, and mix it with loading buffer, boil at 100° C. for 10 minutes and freeze at −20° C. for later western blot testing.

(10) Western blot test to identify protein knockout: prepare protein gel according to the instruction of Yamei PAGE Gel Rapid Preparation Kit (10%), add sample to the loading well and add 2.5-6 μL of protein marker (Thermo 26616) to the left and right lanes of the sample, keep 60V for 30 min in the stacking gel, keep 90V for 100 min in the separation gel, and after electrophoresis, transfer the protein to the membrane according to the wet transfer method (80V, 120 min). After the transfer is completed, cut the membrane according to the size of the target protein. The target protein strip after cutting is placed and sealed in 5% milk for 30 minutes. Wash with TBST twice (10 min/time), and add 4 mL of primary antibody incubation solution (1:1500) and incubate overnight on a shaker at 4° C.; wash with TBST 3 times (10 min/time), add 4 mL of secondary antibody incubation solution (1:15000) and incubate at room temperature for 120 min, wash with TBST 3 times (10 min/time) and then perform protein exposure.

A PD1 knockout cell line is successfully established based on the PE-STOP method, as shown in FIG. 4B, and Western blot testing is used to identify the complete knockout of the protein, as shown in FIG. 4C.

### Embodiment 3. Comparison of Different Knockout Schemes in HEK 293T Cells

Taking the human genome as an example, calculate the termination codon introduction coverage of different gene knockout schemes (PE-STOP vs. iSTOP and i-Silence), randomly select a certain number of genes, and design corresponding pegRNA and sgRNA, based on the above method, introduce termination codons and calculate the purity of the editing products, and identify off-target phenomena at the DNA level and RNA level of different methods through website prediction and whole transcriptome sequencing.

### Statistics on the Coverage of Different Gene Knockout Strategies in the Human Genome

### 1. Statistics of ORF Coverage

(1) Extract the reference gene sequences of the human genome from the NCBI database, remove pseudogenes and non-coding RNA sequences, keep the coding gene sequences, search for the start codon coding sequence (ATG), and search for the termination codon coding sequences (TAG, TGA, TAA), define a complete sequence with a start codon and a termination codon as an ORF sequence, and extract all ORF sequences as a new set for the next calculation of the number of editing sites.

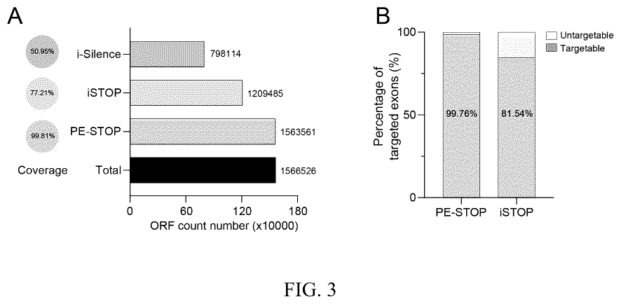

(2) Set the editing motif of PE-STOP, iSTOP and i-Silence, and use this as a basis to calculate the coverage of various methods in the ORF set extracted in the previous step. The editing motifs of PE-STOP are: NNN(−14˜−1)NNNNGG and CCNNNN(−1˜−14)NNN; the editing motifs of iSTOP are: CAA/CAG/CGAN(10˜14)NGG and CCNN(10-15)TGG; the editing motifs of i-Silence are: ATGN(10-14)NGG and CCNN(11-15)TAC. The calculation results are shown in FIG. 3A.

### 2. Statistics on the Coverage of Exon

(1) Extract the reference gene sequences of the human genome from the NCBI database, remove pseudogenes and non-coding RNA sequences, keep the coding gene sequences, and extract all exon sequences according to NCBI annotations as a new set for the next calculation of the number of editing sites.

(2) Set the editing motif of PE-STOP, iSTOP and i-Silence, and use this as a basis to calculate the coverage of various methods in the exon set extracted in the previous step. The editing motifs of PE-STOP are: NNN(−14˜−1)NNNNGG and CCNNNN(−1˜−14)NNN; the editing motifs of iSTOP are: CAA/CAG/CGAN(10˜14)NGG and CCNN(10-15)TGG; the editing motifs of i-Silence are: ATGN(10-14)NGG and CCNN(11-15)TAC. The calculation results are shown in FIG. 3B.

### II. Preparation of Components of Different Editing Strategies Targeting the Target Site of HEK 293T Cells and Detection of Editing Specificity

1. Prepare pegRNA and Nick sgRNA Vectors Targeting Different Sites of PE-STOP

(1) Design 10 pegRNA vector sequences targeting 5 genes (HNRNPK, DKC1, HPRT1, CTNNB1, HSD17B4)

TABLE 7
Preparation of spacer and RT-PBS oligonucleotide
sequences targeting target sites

| | | Oligo- | oligo- |
|---|---|---|---|
| Target | pegRNA | nucleotide | nucleotide |
| gene | component | chain name | chain sequence |
| HPRT1-site1 | Spacer | Forward59 | accgCGCGCC |
| | | | GGCCGGCTCC |
| | | | GTTAgtttc |
| | | Reverse60 | ctctgaaacT |
| | | | AACGGAGCCG |
| | | | GCCGGCGCG |
| | PBSRT | Forward61 | gtgcCGGAGC |
| | | | CGGCCGGGCG |
| | | | GGTCGCCACA |
| | | | A |
| | | Reverse62 | gacaTTGTGG |
| | | | CGACCCGCCC |
| | | | GGCCGGCTCC |
| | | | G |
| HPRT1-site2 | Spacer | Forward63 | accgTCTTGC |
| | | | TCGAGATGTG |
| | | | ATGAgtttc |
| | | Reverse64 | ctctgaaacT |
| | | | CATCACATCT |
| | | | CGAGCAAGA |
| | PBSRT | Forward65 | gtgcTCACAT |
| | | | CTCGAGCTCC |
| | | | CATCTCTCAC |
| | | | A |
| | | Reverse66 | gacaTGTGAG |
| | | | AGATGGGAGC |
| | | | TCGAGATGTG |
| | | | A |
| CTNNB1-site1 | Spacer | Forward67 | accgCCACTC |
| | | | ATACAGGACT |
| | | | TGGGgtttc |
| | | Reverse68 | ctctgaaacC |
| | | | CCAAGTCCTG |
| | | | TATGAGTGG |
| | PBSRT | Forward69 | gtgcAAGTCC |
| | | | TGTATGAAGG |
| | | | ATGTGGATAC |
| | | | CTGAC |
| | | Reverse70 | gacaGTCAGG |
| | | | TATCCACATC |
| | | | CTTCATACAG |
| | | | GACTT |
| CTNNB1-site2 | Spacer | Forward71 | accgATGCAA |
| | | | TGACTCGAGC |
| | | | TCAGgtttc |
| | | Reverse72 | ctctgaaacC |
| | | | TGAGCTCGAG |
| | | | TCATTGCAT |
| | PBSRT | Forward73 | gtgcAGCTCG |
| | | | AGTCATTAGC |
| | | | TCGTACCCTC |
| | | | TA |
| | | Reverse74 | gacaTAGAGG |
| | | | GTACGAGCTA |
| | | | ATGACTCGAG |
| | | | CT |
| DKC1-site1 | Spacer | Forward75 | accgCTAAGT |
| | | | TGGACACGTC |
| | | | TCAGgtttc |
| | | Reverse76 | ctctgaaacC |
| | | | TGAGACGTGT |
| | | | CCAACTTAG |
| | PBSRT | Forward77 | gtgcAGACGT |
| | | | GTCCAACCAA |
| | | | AAGGGGCCAC |
| | | | TA |
| | | Reverse78 | gacaTAGTGG |
| | | | CCCCTTTTGG |
| | | | TTGGACACGT |
| | | | CT |
| DKC1-site2 | Spacer | Forward79 | accgGACCTA |
| | | | AACCCCACTT |
| | | | CCGAgtttc |
| | | Reverse80 | ctctgaaacT |
| | | | CGGAAGTGGG |
| | | | GTTTAGGTC |
| | PBSRT | Forward81 | gtgcGAAGTG |
| | | | GGGTTTAAGA |
| | | | CACTTACCCT |
| | | | TA |
| | | Reverse82 | gacaTAAGGG |
| | | | TAAGTGTCTT |
| | | | AAACCCCACT |
| | | | TC |
| HNRNPK-site1 | Spacer | Forward83 | accgATTGGT |
| | | | TTCAGTGTTA |
| | | | GGGAgtttc |
| | | Reverse84 | ctctgaaacT |
| | | | CCCTAACACT |
| | | | GAAACCAAT |
| | PBSRT | Forward85 | gtgcCTAACA |
| | | | CTGAAACCCA |
| | | | GAAGAAACCT |
| | | | GAC |
| | | Reverse86 | gacaGTCAGG |
| | | | TTTCTTCTGG |
| | | | GTTTCAGTGT |
| | | | TAG |
| HNRNPK-site2 | Spacer | Forward87 | accgCCTCTA |
| | | | GGTGGTGGTG |
| | | | GTGGgtttc |
| | | Reverse88 | ctctgaaacC |
| | | | CACCACCACC |
| | | | ACCTAGAGG |
| | PBSRT | Forward89 | gtgcCCACCA |
| | | | CCACCTAATC |
| | | | TTCCTCTTCC |
| | | | TTAA |
| | | Reverse90 | gacaTTAAGG |
| | | | AAGAGGAAGA |
| | | | TTAGGTGGTG |
| | | | GTGG |
| HSD17B4-site1 | Spacer | Forward91 | accgACCCGC |
| | | | CCGTCGAACC |
| | | | TCAGgtttc |
| | | Reverse92 | ctctgaaacC |
| | | | TGAGGTTCGA |
| | | | CGGGCGGGT |
| | PBSRT | Forward93 | gtgcAGGTTC |
| | | | GACGGGCATG |
| | | | GGCTCACCGT |
| | | | AG |
| | | Reverse94 | gacaCTACGG |
| | | | TGAGCCCATG |
| | | | CCCGTCGAAC |
| | | | CT |
| HSD17B4-site2 | Spacer | Forward95 | accgACTCAG |
| | | | ACAGTTATGC |
| | | | CTGAgtttc |
| | | Reverse96 | ctctgaaacT |
| | | | CAGGCATAAC |
| | | | TGTCTGAGT |
| | PBSRT | Forward97 | gtgcGGCATA |
| | | | ACTGTCTTGC |
| | | | TTACTTACCT |
| | | | TAA |
| | | Reverse98 | gacaTTAAGG |
| | | | TAAGTAAGCA |
| | | | AGACAGTTAT |
| | | | GCC |

The specific preparation process of the PegRNA vector is completely consistent with the process in Embodiment 1. The prepared vector is frozen at −20° C. for later use.

(2) Design 10 nick sgRNA vector sequences targeting 5 genes (HNRNPK, DKC1, HPRT1, CTNNB1, HSD17B4)

TABLE 8
Preparation of nick sgRNA oligonucleotide
sequences targeting target sites

| | Oligo- | |
|---|---|---|
| Target | nucleotide | |
| gene | chain name | Sequence |
| HPRT1-sg1 | Forward99 | accgCACTGC |
| | | GGATCCCGCG |
| | | CCTC |
| | Reverse100 | aaacGAGGCG |
| | | CGGGATCCGC |
| | | AGTG |
| HPRT1-sg2 | Forward101 | accgGTGCTT |
| | | TGATGTAATC |
| | | CAGC |
| | Reverse102 | aaacGCTGGA |
| | | TTACATCAAA |
| | | GCAC |
| CTNNB1-sg1 | Forward103 | accgACCACA |
| | | GCTCCTTCTC |
| | | TGAG |
| | Reverse104 | aaacCTCAGA |
| | | GAAGGAGCTG |
| | | TGGT |
| CTNNB1-sg2 | Forward105 | accgGCAGCA |
| | | TCAAACTGTG |
| | | TAGA |
| | Reverse106 | aaacTCTACA |
| | | CAGTTTGATG |
| | | CTGC |
| DKC1-sg1 | Forward107 | accgCTTGGA |
| | | AATAACGTAA |
| | | AAGC |
| | Reverse108 | aaacGCTTTT |
| | | ACGTTATTTC |
| | | CAAG |
| DKC1-sg2 | Forward109 | accgGTCATC |
| | | TCTACCTGCG |
| | | ACCA |
| | Reverse110 | aaacTGGTCG |
| | | CAGGTAGAGA |
| | | TGAC |
| HNRNPK-sg1 | Forward111 | accgGCCCGT |
| | | TTAATAAAAG |
| | | AATA |
| | Reverse112 | aaacTATTCT |
| | | TTTATTAAAC |
| | | GGGC |
| HNRNPK-sg2 | Forward113 | accgGCCGGG |
| | | GTGGTAGCAG |
| | | AGCT |
| | Reverse114 | aaacAGCTCT |
| | | GCTACCACCC |
| | | CGGC |
| HSD17B4-sg1 | Forward115 | accgGTTCGT |
| | | GTGTGTGTCG |
| | | TTGC |
| | Reverse116 | aaacGCAACG |
| | | ACACACACAC |
| | | GAAC |
| HSD17B4-sg2 | Forward117 | accgTTGTAA |
| | | AGCTCATTCC |
| | | ACAT |
| | Reverse118 | aaacATGTGG |
| | | AATGAGCTTT |
| | | ACAA |

The specific process of preparing Nick sgRNA vector is completely consistent with the process in Embodiment 1. The prepared vector is frozen at −20° C. for later use.
2. Preparation of sgRNA Vectors Targeting Different Sites of iSTOP and i-Silence

(1) Design 20 iSTOP sgRNA vector sequences targeting 5 genes (HNRNPK, DKC 1, HPRT1, CTNNB1, HSD17B4)

TABLE 9
Preparation of iSTOP sgRNA oligonucleotide
sequences targeting target sites

| | Oligo- | |
|---|---|---|
| Target | nucleotide | |
| gene | chain name | Sequence |
| HPRT1-CBE-sg1 | Forward119 | accgTCTTGC |
| | | TCGAGATGTG |
| | | ATGA |
| | Reverse120 | aaacTCATCA |
| | | CATCTCGAGC |
| | | AAGA |
| HPRT1-CBE-sg2 | Forward121 | accgAATGCA |
| | | GACTTTGCTT |
| | | TCCT |
| | Reverse122 | aaacAGGAAA |
| | | GCAAAGTCTG |
| | | CATT |
| HPRT1-CBE-sg3 | Forward123 | accgCAGGCA |
| | | GTATAATCCA |
| | | AAGA |
| | Reverse124 | aaacTCTTTG |
| | | GATTATACTG |
| | | CCTG |
| CTNNB1-CBE-sg1 | Forward125 | accgCTGGCA |
| | | GCAACAGTCT |
| | | TACC |
| | Reverse126 | aaacGGTAAG |
| | | ACTGTTGCTG |
| | | CCAG |
| CTNNB1-CBE-sg2 | Forward127 | accgTACCCA |
| | | GCGCCGTACG |
| | | TCCA |
| | Reverse128 | aaacTGGACG |
| | | TACGGCGCTG |
| | | GGTA |
| CTNNB1-CBE-sg3 | Forward129 | accgACACAG |
| | | CAGCAATTTG |
| | | TGGT |
| | Reverse130 | aaacACCACA |
| | | AATTGCTGCT |
| | | GTGT |
| CTNNB1-CBE-sg4 | Forward131 | accgCCTCCC |
| | | AAGTCCTGTA |
| | | TGAG |
| | Reverse132 | aaacCTCATA |
| | | CAGGACTTGG |
| | | GAGG |
| CTNNB1-CBE-sg5 | Forward133 | accgACATCA |
| | | AGAAGGAGCT |
| | | AAAA |
| | Reverse134 | aaacTTTTAG |
| | | CTCCTTCTTG |
| | | ATGT |
| CTNNB1-CBE-sg6 | Forward135 | accgCTGCCA |
| | | AGTGGGTGGT |
| | | ATAG |
| | Reverse136 | aaacCTATAC |
| | | CACCCACTTG |
| | | GCAG |
| DKC1-CBE-sg1 | Forward137 | accgCACAAC |
| | | AGAGTGCAGG |
| | | TATG |
| | Reverse138 | aaacCATACC |
| | | TGCACTCTGT |
| | | TGTG |
| DKC1-CBE-sg2 | Forward139 | accgGTGGTC |
| | | AGATGCAGGA |
| | | GCTT |
| | Reverse140 | aaacAAGCTC |
| | | CTGCATCTGA |
| | | CCAC |
| DKC1-CBE-sg3 | Forward141 | accgGATTCG |
| | | ACGGATACTT |
| | | CGGG |
| | Reverse142 | aaacCCCGAA |
| | | GTATCCGTCG |
| | | AATC |
| DKC1-CBE-sg4 | Forward143 | accgGCGGCG |
| | | AGTTGTTTAC |
| | | CCTT |
| | Reverse144 | aaacAAGGGT |
| | | AAACAACTCG |
| | | CCGC |
| HNRNPK-CBE-sg1 | Forward145 | accgATTCAT |
| | | CAGAGTCTAG |
| | | CAGG |
| | Reverse146 | aaacCCTGCT |
| | | AGACTCTGAT |
| | | GAAT |
| HNRNPK-CBE-sg2 | Forward147 | accgCCCGGA |
| | | CGAGGCGGCC |
| | | GGGG |
| | Reverse148 | aaacCCCCGG |
| | | CCGCCTCGTC |
| | | CGGG |
| HNRNPK-CBE-sg3 | Forward149 | accgTAAACA |
| | | AATCCGTCAT |
| | | GAGT |
| | Reverse150 | aaacACTCAT |
| | | GACGGATTTG |
| | | TTTA |
| HSD17B4-CBE-sg1 | Forward151 | accgACTCAG |
| | | ACAGTTATGC |
| | | CTGA |
| | Reverse152 | aaacTCAGGC |
| | | ATAACTGTCT |
| | | GAGT |
| HSD17B4-CBE-sg2 | Forward153 | accgATAGGT |
| | | CAGAAATCTA |
| | | TGAT |
| | Reverse154 | aaacATCATA |
| | | GATTTCTGAC |
| | | CTAT |
| HSD17B4-CBE-sg3 | Forward155 | accgGTGTAC |
| | | CAAGGCCCTG |
| | | CAAA |
| | Reverse156 | aaacTTTGCA |
| | | GGGCCTTGGT |
| | | ACAC |
| HSD17B4-CBE-sg4 | Forward157 | accgTCTACA |
| | | AACTGAGATG |
| | | TGGA |
| | Reverse158 | aaacTCCACA |
| | | TCTCAGTTTG |
| | | TAGA |

(2) Design three i-Silence sgRNA vector sequences targeting three genes (DKC1, HPRT1, HSD17B4)

TABLE 10
Preparation of i-Silence sgRNA oligo-
nucleotide sequences targeting target
sites

| | Oligo- | |
|---|---|---|
| Target | nucleotide | |
| gene | chain name | Sequence |
| HPRT1- | Forward159 | accgGTTATGGCGACCCGCAGCCC |
| ABE-sg | | |
| | Reverse160 | aaacGGGCTGCGGGTCGCCATAAC |
| DKC1- | Forward161 | accgGGTAACATGGCGGATGCGGA |
| ABE-sg | | |
| | Reverse162 | aaacTCCGCATCCGCCATGTTACC |
| HSD17B4- | Forward163 | accgTATTCATGGGCTCACCGCTG |
| ABE-sg | | |
| | Reverse164 | aaacCAGCGGTGAGCCCATGAATA |

(3) Anneal the sgRNA oligonucleotide chain of iSTOP and i-Silence to obtain the sgRNA annealing product. The annealing system and procedure are as follows:

### Annealing System:

| | |
|---|---|
| Forward (100 μM) | 5 μL |
| Reverse (100 μM) | 5 μL |
| Total | 10 μL |

### Annealing Procedure:

| | | |
|---|---|---|
| 95° C. | 5 | min |
| 98° C. to 8° C. | −2° | C./cycle |
| 85° C. to 2° C. | −0.1° | C./cycle |

(4) Digest pGL3-U6-sgRNA-mCherry using BsaI restriction endonuclease at 37° C. overnight. The digestion system is as follows:

| | | |
|---|---|---|
| pGL3-U6-sgRNA-mCherry | 2000 | ng |
| 10 × CutSmart Buffer | 5 | μL |
| Bsal-HFV2 | 1 | μL |
| ddH₂O | Add to 50 | μL |

(5) Purify and recover the linearized pGL3-U6-sgRNA-mCherry, and ligate it with the sgRNA annealing product at 16° C. overnight. The ligation system is as follows:

| | |
|---|---|
| Solution I | 5 μL |
| sgRNA annealing product | 4 μL |
| Digested pGL3-U6-sgRNA-mCherry | 1 μL |

(6) Transform the ligation product into DH5α competent cells, and select single clones for Sanger sequencing the next day. Extract plasmids from single clone colonies with correct sequencing results to obtain targeting sgRNA expression plasmids of iSTOP and i-Silence.

### 3. Statistics on Cell Transfection and Editing Specificity

(1) Preparation of HEK 293T cells: resuscitate the frozen cells into a 10 cm culture dish, add 10 mL DMEM culture medium (10% serum+working concentration of streptomycin/penicillin) for culture, perform cell planking or passage after until the cell confluence reaches 80˜90%.

(2) Preparation of cells in a 24-well plate: the cells in a 10 cm culture dish with a cell confluence of 80% are planked (plated) in a 24-well plate at a ratio of 1:3. When planking (plating), add 300 μL of culture solution to each well in advance, add 200 μL of centrifuged resuspended cells to each well, shake evenly using a cross method, and place them in the incubator for 12-24 hours.

(3) After 24 hours of adherent growth of the cells in the 24-well plate, observe the cell confluence under a microscope, carry out transfection when the confluence reaches 60%˜80%. The plasmids and related ratios for transfection are as follows: PE-STOP (the system as set forth in Embodiment 1), iSTOP (AncBE4max 900 ng+sgRNA 300 ng), i-Silence (ABE8e 900 ng+sgRNA 300 ng), the transfection reagent is EZ trans (2.5 μL), and the specific transfection steps are the same as Embodiment 1.

(4) After 24 hours of transfection, replace each well with 1 mL of fresh culture solution and continue to culture until 72 hours. The digested cells are passed through a cell sieve into a flow tube for flow sorting. 20,000 mCherry (+) cells are sorted to into a 1.5 mL EP tube, centrifuge at 12000 rpm for 2 min, discard the supernatant, and add 40 μL of cell lysis buffer for lysis. Place them at 37° C. for 1 hour and at 80° C. for 30 minutes.

(5) Take 3 μL of lysis buffer and perform PCR amplification of the target gene segment.

### The PCR Reaction System and Condition are as Follows:

| | | |
|---|---|---|
| Buffer | 12.5 | μL |
| dNTP Mix | 0.5 | μL |
| Primer F | 0.5 | μL |
| Primer R | 0.5 | μL |
| Phanta Super-Fidelity DNA Polymerase | 0.5 | μL |
| cell lysis buffer | 3 | μL |
| ddH₂O | Add to 25 | μL |

### PCR Procedure:

| | | | | |
|---|---|---|---|---|
| 95° C. | 5 | min | | |
| 95° C. | 15 | s | | |
| 68° C. | −0.2° | C./cycle | | |
| 72° C. | 10 | s | {close oversize brace} | 30 cycles |
| 72° C. | 5 | min | | |

| | |
|---|---|
| 4° C. | Forever |

(6) The PCR product is purified and recovered after the band specificity is detected by agarose gel electrophoresis, and then identified by deep targeted sequencing.

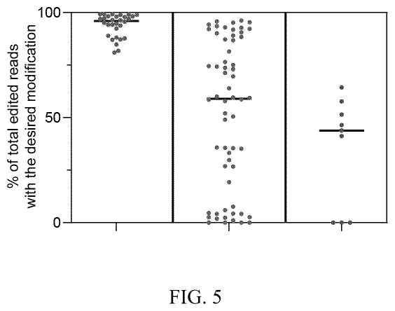

(7) Based on the sequencing results, calculate the editing specificity of different editing methods at the target site. The calculation formula is as follows: the number of reads with only target mutations in the spacer region/the number of all reads carrying target mutations. The statistical results are shown in FIG. 5.

### III. Off-Target Analysis of Different Editing Strategies Targeting HEK 293T Cell Target Sites

### 1. Statistics of Off-Target Activities of Different Methods at DNA Level Based on Website Prediction

Use CasOFFinder website (CRISPR RGEN Tools (rgenome.net)) to performing off-target prediction on pegRNA, nick sgRNA (DKC1-sg1 and HSD17B4-sg1), iSTOP sgRNA (DKC1-CBE-sg1 and HSD17B4-CBE-sg1) and i-Silence sgRNA (DKC1-ABE and HSD17B4-ABE) targeting DKC1 and HSD17B4 (DKC1-site1 and HSD17B4-site1). The predicted off-target sites are shown in the table below:

(1) Prediction of off-target sites of pegRNA targeting HSD17B4

TABLE 11
Prediction of PE-STOP pegRNA-HSD17B4
off-target sites

| Poten- | | | | | |
|---|---|---|---|---|---|
| tial | Sequence | Chromo | -Loca- | | Mismatch |
| sites | information | some | tion | Strand | number |
| OT-1 | ACCCGCCCGTg | chr1 | 1975686 | − | 3 |
| | GAAgCTCcGCG | | | | |
| | G | | | | |
| OT-2 | CCCCGCCCGTC | chr5 | 178164246 | − | 4 |
| | acgCCTCAGTG | | | | |
| | G | | | | |
| OT-3 | AgCCGCCCcTC | chr1 | 167640273 | − | 4 |
| | GAgCCTtAGTG | | | | |
| | G | | | | |
| OT-4 | ACCCtCCCcTa | chr17 | 74179515 | + | 4 |
| | GAgCCTCAGAG | | | | |
| | G | | | | |
| OT-5 | ACCtGCCCcTg | chr17 | 77034471 | − | 4 |
| | GAgCCTCAGTG | | | | |
| | G | | | | |
| OT-6 | ACCCtCCCcTt | chr6 | 89669813 | − | 4 |
| | GAAaCTCAGGG | | | | |
| | G | | | | |
| OT-7 | ACCttCCCaTC | chr6 | 98469360 | − | 4 |
| | aAACCTCAGGG | | | | |
| | G | | | | |
| OT-8 | ACCtGCCCGca | chr10 | 29499798 | − | 4 |
| | GAACaTCAGTG | | | | |
| | G | | | | |

(2) Prediction of off-target sites of pegRNA targeting DKC1

TABLE 12
Prediction of PE-STOP pegRNA-
DKC1 off-target sites

| Poten- | | | | | |
|---|---|---|---|---|---|
| tial | Sequence | Chromo | -Loca- | | Mismatch |
| sites | information | some | tion | Strand | number |
| OT-1 | CTCAGTTGGACA | chr8 | 96056053 | + | 2 |
| | CtTCTCAGTGG | | | | |
| OT-2 | CTgAGTTGGtCA | chr5 | 24649687 | − | 3 |
| | CGTCcCAGGGG | | | | |
| OT-3 | CTgAGTTGGtCA | chr9 | 94250629 | − | 3 |
| | CGTCTCAcGGG | | | | |
| OT-4 | CTcAGTTGtgCA | chr6 | 25683401 | + | 3 |
| | CGTCTCAGAGG | | | | |
| OT-5 | CTgAGTTGGtCA | chr8 | 111931127 | − | 4 |
| | gGTCTCAtGGG | | | | |
| OT-6 | CcAgGTTGGgCA | chr8 | 144472447 | − | 4 |
| | CGTCcCAGTGG | | | | |
| OT-7 | CTgAGTgtGACA | chr15 | 80394606 | − | 4 |
| | tGTCTCAGAGG | | | | |
| OT-8 | CTcAGggGGACA | chr15 | 89357715 | + | 4 |
| | aGTCTCAGGGG | | | | |

(3) Prediction of off-target sites of nick sgRNA targeting HSD17B4

TABLE 13
Prediction of PE-STOP nick sgRNA-
HSD17B4 off-target sites

| Poten- | | | | | |
|---|---|---|---|---|---|
| tial | Sequence | Chromo | -Loca- | | Mismatch |
| sites | information | some | tion | Strand | number |
| OT-1 | GTgtGTGTGTG | chr5 | 176745784 | + | 3 |
| | TGTCGaTGCAG | | | | |
| | G | | | | |
| OT-2 | GTgtGTGTGTG | chr1 | 151979176 | + | 3 |
| | TGTCtTTGCAG | | | | |
| | G | | | | |
| OT-3 | cTTCtTGTGTG | chr7 | 133784506 | + | 3 |
| | TGgCGTTGCTG | | | | |
| | G | | | | |
| OT-4 | GTTCtTGgGTG | chr12 | 103007257 | − | 3 |
| | TGTCtTTGCAG | | | | |
| | G | | | | |

(4) Prediction of off-target sites of nick sgRNA targeting DKC1

TABLE 14
Prediction of PE-STOP nick sgRNA-
DKC1 off-target sites

| Poten- | | | | | |
|---|---|---|---|---|---|
| tial | Sequence | Chromo | -Loca- | | Mismatch |
| sites | information | some | tion | Strand | number |
| OT-1 | CTTGGAAATA | chr4 | 102221844 | − | 3 |
| | ACGTcgAgGC | | | | |
| | TGG | | | | |
| OT-2 | CTTtcAAATA | chr17 | 14076630 | + | 3 |
| | AtGTAAAAGC | | | | |
| | TGG | | | | |
| OT-3 | CTTGGAAATc | chr13 | 28338064 | − | 3 |
| | AgtTAAAAGC | | | | |
| | TGG | | | | |
| OT-4 | CTTGGAAATA | chr3 | 154484273 | − | 3 |
| | ACtTAAAAtt | | | | |
| | TGG | | | | |
| OT-5 | CaaGGAAATA | chr3 | 184624932 | − | 3 |
| | ACcTAAAAGC | | | | |
| | GGG | | | | |
| OT-6 | aTTGaAAAgA | chr8 | 21890592 | − | 4 |
| | ACGTAAAtGC | | | | |
| | TGG | | | | |
| OT-7 | CTTGaAAcTA | chr8 | 36690361 | + | 4 |
| | AaGTAcAAGC | | | | |
| | AGG | | | | |
| OT-8 | CTTaGAAAgA | chr8 | 50552632 | − | 4 |
| | tCGTAAAAaC | | | | |
| | AGG | | | | |

(5) Prediction of off-target sites of iSTOP-sgRNA targeting HSD17B4

TABLE 15
Prediction of iSTOP-sgRNA-
HSD17B4 off-target sites

| Poten- | | | | | |
|---|---|---|---|---|---|
| tial | Sequence | Chromo | -Loca- | | Mismatch |
| sites | information | some | tion | Strand | number |
| OT-1 | ACaCAcACcG | chr8 | 29927297 | − | 4 |
| | TTAgGCCTGA | | | | |
| | AGG | | | | |
| OT-2 | ACTCAGACAG | chr8 | 79579332 | − | 4 |
| | TTCTGgCTct | | | | |
| | AGG | | | | |
| OT-3 | AaaCAGACAG | chr8 | 119044011 | + | 4 |
| | gaATGCCTGA | | | | |
| | AGG | | | | |
| OT-4 | ACaCtGACAG | chr8 | 129703065 | + | 4 |
| | gTATGCCTGc | | | | |
| | AGG | | | | |
| OT-5 | AtTaAGACAG | chr8 | 135628977 | + | 4 |
| | TTtaGCCTGA | | | | |
| | AGG | | | | |
| OT-6 | AaTCAGACAG | chr8 | 143833817 | + | 4 |
| | TTtaGCaTGA | | | | |
| | GGG | | | | |
| OT-7 | ACaCAcACAG | chr15 | 22723972 | + | 4 |
| | TTATGCtTcA | | | | |
| | TGG | | | | |
| OT-8 | ggTCAGACAG | chr15 | 24774469 | + | 4 |
| | TTATGgCTtA | | | | |
| | GGG | | | | |

(6) Prediction of off-target sites of iSTOP-sgRNA targeting DKC1

TABLE 16
Prediction of iSTOP-sgRNA-
DKC1 off-target sites

| Poten- | | | | | |
|---|---|---|---|---|---|
| tial | Sequence | Chromo | -Loca- | | Mismatch |
| sites | information | some | tion | Strand | number |
| OT-1 | gAaAACAGAG | chr8 | 139863883 | + | 3 |
| | TGCAGGTCTG | | | | |
| | AGG | | | | |
| OT-2 | CACAAggGAG | chr7 | 152746887 | + | 3 |
| | aGCAGGTATG | | | | |
| | AGG | | | | |
| OT-3 | CACAtCAGAG | chr2 | 20340538 | + | 3 |
| | TcCAGGTAgG | | | | |
| | AGG | | | | |
| OT-4 | gACAACAcAG | chr12 | 3923917 | + | 3 |
| | TGCAGGcATG | | | | |
| | TGG | | | | |
| OT-5 | aACAAgAaAG | chr11 | 118509185 | + | 3 |
| | TGCAGGTATG | | | | |
| | TGG | | | | |
| OT-6 | CACAACAGgG | chr8 | 15404343 | + | 4 |
| | TaCAGGaAgG | | | | |
| | TGG | | | | |
| OT-7 | CtCAACAcAG | chr8 | 20936916 | − | 4 |
| | TGCAGGTAct | | | | |
| | GGG | | | | |
| OT-8 | CcCAgCAGAG | chr8 | 50921706 | − | 4 |
| | TGCAGaTATt | | | | |
| | AGG | | | | |

(7) Prediction of off-target sites of i-Silence-sgRNA targeting HSD17B4

TABLE 17
Prediction of i-Silence-sgRNA-
HSD17B4 off-target sites

| Poten- | | | | | |
|---|---|---|---|---|---|
| tial | Sequence | Chromo | -Loca- | | Mismatch |
| sites | information | some | tion | Strand | number |
| OT-1 | TATTCATtGG | chr14 | 78445996 | + | 3 |
| | CTCACaGCTt | | | | |
| | CGG | | | | |
| OT-2 | TATTCATGGG | chr8 | 19698047 | + | 4 |
| | agCtCCaCTG | | | | |
| | CGG | | | | |
| OT-3 | TATTCAaGGa | chr8 | 33130889 | − | 4 |
| | CTCACaGCTa | | | | |
| | GGG | | | | |
| OT-4 | gATTCATGaG | chr8 | 41865188 | + | 4 |
| | CaCACgGCTG | | | | |
| | AGG | | | | |
| OT-5 | TcTTCATGGG | chr8 | 75106138 | − | 4 |
| | CTCAtgGaTG | | | | |
| | GGG | | | | |
| OT-6 | TATTCATaGG | chr15 | 53264963 | − | 4 |
| | CTCACaaCTt | | | | |
| | GGG | | | | |
| OT-7 | TATTCATGcc | chr15 | 53236063 | − | 4 |
| | CTCACtGgTG | | | | |
| | AGG | | | | |
| OT-8 | CATTCATGGa | chr15 | 59135465 | − | 4 |
| | CTCcCCGCaG | | | | |
| | TGG | | | | |

(8) Prediction of off-target sites of i-Silence-sgRNA targeting DKC1

TABLE 18
Prediction of i-Silence-sgRNA-
DKC1 off-target sites

| Poten- | | | | | |
|---|---|---|---|---|---|
| tial | Sequence | Chromo | -Loca- | | Mismatch |
| sites | information | some | tion | Strand | number |
| OT-1 | GGTgACATGG | chr5 | 79955889 | + | 4 |
| | CaGATGCcGg | | | | |
| | GGG | | | | |
| OT-2 | GGTAACATGG | chr5 | 93234740 | + | 4 |
| | tGGtTGCcaA | | | | |
| | GGG | | | | |
| OT-3 | cccAACATGG | chr5 | 113229156 | + | 4 |
| | CGGATGgGGA | | | | |
| | GGG | | | | |
| OT-4 | aGTtACcTGG | chr5 | 178351297 | + | 4 |
| | CGGATGaGGA | | | | |
| | CGG | | | | |
| OT-5 | GGTcAgATGG | chr20 | 42426833 | + | 4 |
| | CtGATGtGGA | | | | |
| | GGG | | | | |
| OT-6 | GGTAACATGG | chr1 | 34685163 | − | 4 |
| | aGGAccCtGA | | | | |
| | AGG | | | | |
| OT-7 | GGTAACAgGt | chr1 | 40662867 | + | 4 |
| | tGGAaGCGGA | | | | |
| | AGG | | | | |
| OT-8 | GtTAACAgGG | chr1 | 57300413 | + | 4 |
| | gGGATGCaGA | | | | |
| | CGG | | | | |

(9) Identification of off-target sites: design corresponding primers for the sequences where the potential off-target sites are located, and use the cell lysate (cell lysis buffer) with edited target site as a template for PCR amplification. The amplification procedure is as follows:

The PCR reaction system and condition are as follows:

| | | |
|---|---|---|
| Buffer | 12.5 | μL |
| dNTP Mix | 0.5 | μL |
| Primer F | 0.5 | μL |
| Primer R | 0.5 | μL |
| Phanta Super-Fidelity DNA Polymerase | 0.5 | μL |
| Cell lysate (cell lysis buffer) | 3 | μL |
| ddH₂O | Add to 25 | μL |

### PCR Procedure:

| | | | | |
|---|---|---|---|---|
| 95° C. | 5 | min | | |
| 95° C. | 15 | s | | |
| 72° C. | −0.2° | C./cycle | | |
| 68° C. | 10 | s | {close oversize brace} | 30 cycles |
| 72° C. | 5 | min | | |

| | |
|---|---|
| 4° C. | Forever |

(10) The PCR product is purified and recovered after the band specificity is detected by agarose gel electrophoresis, and then identified by deep targeted sequencing.

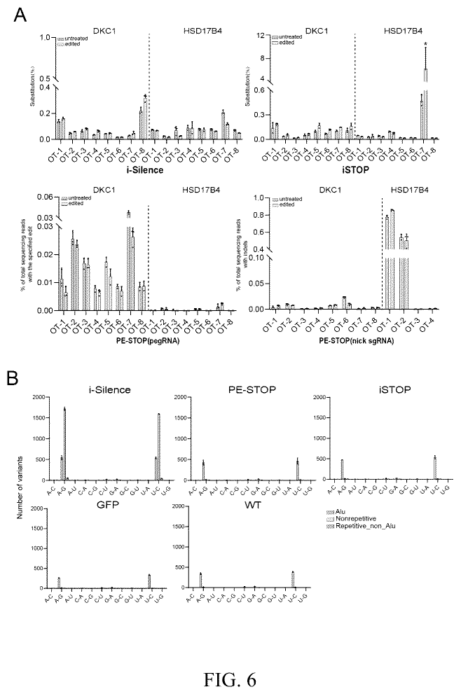

(11) According to the sequencing results, the base mutation frequency in the spacer region is statisticized. The statistical results are shown in FIG. 6A.

### 2. Statistics of Off-Target Activities of Different Methods at the RNA Level Based on Transcriptome Analysis

(1) Prepare RNA sequencing samples, and use different gene termination strategies (PE-STOP, iSTOP, i-Silence) to target DKC1 in HEK293T cells. The specific sequence of pegRNA/sgRNA is: DKC1-site1+DKC1-sg1, DKC1-CBE-sg1, DKC1-ABE.

(2) The process of cell transfection and flow cytometry sorting is the same as the steps in Embodiment 1. The only difference is that the number of collected cells is adjusted to 50,000.

(3) Extraction of total RNA: centrifuge the collected cells at 12000 rpm for 5 minutes, after centrifugation, use a vacuum aspirator to discard the supernatant liquid, add 1 mL of Trizol lysis solution to each sample and pipet (blow and beat) repeatedly until the precipitate disappears, place the samples on ice to lyse for 15 min. Add 200 μL chloroform, invert 5 times until the liquid becomes turbid, then place it on ice for 20 minutes, and then put it into a 4° C. centrifuge for high-speed centrifugation at 12000 rpm for 30 minutes. After centrifugation, the liquid is divided into three layers, take the uppermost liquid into a new enzyme-free tube, add an equal volume of isopropyl alcohol solution, invert 5-8 times, put it into a 4° C. centrifuge for high-speed centrifugation at 12000 rpm for 30 minutes. Discard the supernatant, add 1 mL of 75% ethanol solution to wash the precipitate, and put it into a 4° C. centrifuge for high-speed centrifugation at 12000 rpm for 15 minutes. Aspirate off the ethanol solution, open the lid and let stand for 15 minutes. After the ethanol has evaporated, add 40 μL of enzyme-free water to dissolve the precipitate, use a UV spectrophotometer to measure the RNA concentration. Samples with a total amount higher than 1 μg are sent to the company for RNA library construction and sequencing. The analysis results of RNA sequencing data are shown in FIG. 6B.

The above embodiments show that PE-STOP has much higher coverage in the human genome than iSTOP and i-Silence, and it is verified in two cell lines that the PE-STOP method can efficiently mutate different types of amino acids into premature termination codons and successfully establish the knockout cell line, and the editing specificity and off-target analysis in HEK 293T cells further demonstrates the safety of PE-STOP. The above results strongly illustrate the flexibility of the PE-STOP method and the practicability of establishing knockout cell lines. The establishment of the PE-STOP method promotes the application of guided editing systems and has broad application prospects in the field of gene knockout.

Although the preferred embodiments of the present disclosure have been described, those skilled in the art will be able to make additional changes and modifications to these embodiments once the basic inventive concepts are apparent. Therefore, it is intended that the appended claims are construed to include the preferred embodiments and all changes and modifications that fall within the scope of the present disclosure.

Obviously, those skilled in the art can make various changes and modifications to the present disclosure without departing from the spirit and scope of the present disclosure. In this way, if these changes and modifications of the present disclosure fall within the scope of the claims of the present disclosure and equivalent technologies, the present disclosure is also intended to include these changes and modifications.

This application contains a Sequence Listing XML as a separate part of the disclosure, which presents nucleotide and/or amino acid sequences and associated information using the symbols and format in accordance with the requirements of 37 CFR-1.831-1.835. The XML file named “PE-STOP.xml”, created Jan. 27, 2024, 4000 bytes in size, is submitted herewith and is incorporated by reference in its entirety.

What is claimed is:

1. A PE-STOP gene editing system, comprising a prime editor protein, a pegRNA targeting a target site, and a corresponding nicking sgRNA for cleaving, wherein the prime editor protein is selected from a PEmax protein, and an amino acid sequence of the PEmax is shown in SEQ ID NO.1.

2. The PE-STOP gene editing system according to claim 1, wherein a 3′ end of the pegRNA contains an anti-degradation xrRNA moiety.

3. A method of use of the gene editing system according to claim 1 comprising editing genome sequences of organisms or biological cells.

4. The method according to claim 3, wherein the editing involves base substitution of a target gene sequence and introduction of a termination codon in advance, thereby achieving knockout of a target gene.

5. The method according to claim 4, wherein the number of introduced termination codons is 2-3.

6. The method according to claim 5, wherein an editing position of the target gene sequence comprises an NGG PAM sequence, and the editing position is located in the first 20% of the target gene sequence.

7. The method according to claim 6, wherein a mutant sequence after base substitution comprises TAG, TGA, and/or TAA.

8. A method for efficiently achieving target gene knockout, comprising:
S1: constructing a plasmid containing the gene editing system according to claim 1 based on a target gene sequence,;
S2: introducing the plasmid into a biological cell, performing gene editing on the biological cell, and making a target gene undergo premature termination codon mutation.
